(19) United States
(12) Patent Application Publication
(10) Pub. No.: US 2003/0204120 A1
(43) Pub. Date: Oct. 30, 2003

(54) Selective hydrogenation process

(76) Inventors: Joseph J. Bergmeister, Kingwood, TX (US); David A. Young, Caney, KS (US)

Correspondence Address: CHEVRON PHILLIPS CHEMICAL COMPANY LP, LAW DEPARTMENT - IP, P.O BOX 4910, THE WOODLANDS, TX, 77387-4910, US

(21) Appl. No.: 10/133,150

(22) Filed: Apr. 26, 2002

(51) Int. Cl. C07C 7/163; C07C 7/167

(52) U.S. Cl. 585/258; 585/259; 585/261

(57) ABSTRACT

In a selective hydrogenation process in which highly unsaturated hydrocarbons such as diolefins and/or alkynes are contacted with catalyst compositions containing palladium, an inorganic support and, optionally, a component silver or alkali metal fluoride in the presence of hydrogen to produce less unsaturated hydrocarbons such as monoolefins the catalyst is contacted with a fluid containing sulfur.

### FIELD OF THE INVENTION

This invention relates to the use of a sulfur compound(s) for preventing an uncontrollable hydrogenation reaction (runaway) while selectively hydrogenating a highly unsaturated hydrocarbon.

### BACKGROUND OF THE INVENTION

Ethylene and propylene are produced commercially by steam cracking a saturated hydrocarbon. During the production of the olefins many impurities are also formed, such as, oligimers, alkynes, and dienes. The oligimers are typically removed by distillation, and the alkynes and dienes are usually hydrogenated to their respective olefins in an olefin rich stream. Therefore a selective hydrogenation catalyst must be used so as to hydrogenate the alkyne or diene to an olefin without hydrogenating the olefin(s) to a saturated hydrocarbon. If large amounts of the olefin(s) begin to hydrogenate and the reactor can not dissipate the heat, then the reaction can runaway (an uncontrollable exotherm).

Even though there are many selective hydrogenation catalysts, a sudden upset in the feed stream can trigger a runaway. This is of concern since the reactor temperature could rise several hundred degrees in seconds. For this reason there is the need to make further improvements in the hydrogenation process so as to be able to stop an uncontrollable hydrogenation reaction. Accordingly, the development of a process where a reaction moderator can be added to the hydrogenation reactor that would stop the runaway reaction, would be a significant contribution to the art.

### SUMMARY OF THE INVENTION

It is an object of this invention to employ a sulfur compound in the acetylene hydrogenation unit (also known as the ARU (acetylene removal unit) to stop an uncontrollable hydrogenation reaction. Such reactions can not correct themselves without outside intervention. An advantage of this invention is the prevention of a possible melt down of the hydrogenation unit. Other objects and advantages will become more apparent as the invention is more fully disclosed hereinbelow.

According to this invention, a process which can be used for stopping an uncontrollable hydrogenation in an ARU is provided. The process comprises of adding a small amount of a sulfur compound to the ARU unit in concentrations sufficient to poison the hydrogenation reaction. The sulfur compound can be hydrogen sulfide (H2S) carbonyl sulfide (COS) a mercaptane, or a polysulfide. Preferably the sulfur species is COS.

### DETAILED DESCRIPTION OF THE INVENTION

As used in the present invention, the term “fluid” denotes gas, liquid, or combination thereof. The term “substantial” or “substantially” generally means more than trivial. A “saturated hydrocarbon” is referred to as any hydrocarbon which does not contain any carbon to carbon multiple bonds. Example of saturated hydrocarbons include, but are not limited to, ethane, propane, butanes, pentanes, hexanes, octanes, decanes, naphtha, and combinations of any two or more thereof. An “unsaturated hydrocarbon” as used in this invention is a hydrocarbon having one double bond between carbon atoms in the molecule. Examples of unsaturated hydrocarbons include, but are not limited to, monoolefins such as ethylene, propylene, butenes, pentenes, hexenes, octenes, and decenes. The term “highly unsaturated hydrocarbon” refers to a hydrocarbon which contains a triple bond or two or more double bonds in a molecule. Examples of unsaturated hydrocarbons include, but are not limited to, alkynes such as acetylene, propyne, and butynes; diolefins such as propadiene, butadienes, pentadienes (including isoprene), hexadienes, octadienes, decadienes aromatic compounds such as benzene and naphthalene; and combinations of two or more thereof. The term “less unsaturated hydrocarbon” refer to a hydrocarbon in which the triple bond in the highly unsaturated hydrocarbon is hydrogenated to a double bond or a hydrocarbon in which the number of double bonds is one less, or at least one less, than that in the highly unsaturated hydrocarbon. The term “selective hydrogenation” is referred to as a hydrogenation process which converts a highly unsaturated hydrocarbon such as an alkyne or a diolefin to a less unsaturated hydrocarbon such as a monoolefin without hydrogenating the less unsaturated hydrocarbon to a more saturated hydrocarbon or a saturated hydrocarbon such as alkane.

The term “runaway reaction” refers to the situation whereby the highly unsaturated hydrocarbon is hydrogenated as well as a significant amounts of the unsaturated hydrocarbon(s) such that the reactor can not dissipate the heat. At this point the reactor can not recover on its own and will continue to hydrogenate the unsaturated hydrocarbon until a catastrophic failure occurs.

The catalyst composition useful in this invention comprises of (a) palladium such as palladium metal, palladium oxide, or combinations, (b) a modified inorganic support and, optionally, (c) silver and/or (d) a modifier(s). The composition can have any suitable shape such as spherical, cylindrical, trilobal, or combinations of two or more thereof. These catalysts have been described in the literature and are available commercially.

According to this invention, a selective hydrogenation process is provided. In the selective hydrogenation process of this invention, feed which contains one or more highly unsaturated hydrocarbon(s) is contacted, in the presence of hydrogen, with a catalyst composition as described above. Preferably the feed containing the highly unsaturated hydrocarbon(s) also comprises an unsaturated hydrocarbon(s). The highly unsaturated hydrocarbon is present as an alkyne, a polyene, such as a diolefin, or combinations of two or more thereof, as an impurity, generally at a level of about 1 mg/Kg (ppmw) to about 50,000 ppmw each in the feed. The unsaturated hydrocarbon(s) in the feed can be one or more alkenes.

Examples of suitable alkynes include, but are not limited to, acetylene, propyne, 1-butyne, 2-butyne, 1-pentyne, 2-pentyne, 3-methyl-1-butyne, 1-hexyne, 1-heptyne, 1-octyne, 1-nonyne, 1-decyne, and combinations of two or more thereof. The presently preferred alkynes are acetylene and propyne.

These alkynes are primarily hydrogenated to the corresponding alkenes. For example, acetylene is primarily hydrogenated to ethylene, propyne is primarily hydrogenated to propylene, and the butynes are primarily hydrogenated to the corresponding butenes (1-butene, 2-butenes).

Examples of suitable polyenes include those containing about 3 to about 12 carbon atoms per molecule. The presently preferred polyenes are diolefins. Such diolefins include, but are not limited to, propadiene, 1,2-butadiene, 1,3-butadiene, isoprene, 1,2-pentadiene, 1,3-pentadiene, 1,4-pentadiene, 1,2-hexadiene, 1,3-hexadiene, 1,4-hexadiene, 1,5-hexadiene, 2-methyl-1,2-pentadiene, 2,3-dimethyl-1,3-butadiene, heptadienes, methylhexadienes, octadienes, methylheptadienes, dimethylhexadienes, ethylhexadienes, trimethylpentadienes, methyloctadienes, dimethylheptadienes, ethyloctadienes, trimethylhexadienes, nonadienes, decadienes, undecadienes, dodecadienes, cyclopentadienes, cyclohexadienes, methylcyclopentadienes, cycloheptadienes, methylcyclohexadienes dimethylcyclopentadienes, ethylcyclopentadienes, dicyclopentadiene, and mixtures of one or two of these diolefins. Presently preferred diolefins are propadiene, 1,2-butadiene, pentadienes (such as 1,3-pentadiene, 1,4-pentadiene, isoprene), cyclopentadienes (such as 1,3-cyclopentadiene) and dicyclopentadiene (also known as tricyclo[5.2.1]²⁶ deca-3,8-diene). These diolefins are preferably hydrogenated to their corresponding monoolefins containing the same number of carbon atoms per molecule as the diolefins. For example, propadiene is hydrogenated to propylene, 1,3-butadiene is hydrogenated to 1-butene and 2-butene, 1,3-pentadiene and 1,4-pentadiene are hydrogenated to 1-pentene and 2-pentene, isoprene is hydrogenated to methyl-1-pentenes and methyl-2-pentenes, and 1,3-cyclopentadiene is hydrogenated to cyclopentene.

The highly unsaturated hydrocarbon-containing feed for the hydrogenation process of this invention can also comprise one or more additional hydrocarbons, in particular, monoolefins, aromatic hydrocarbons, and saturated hydrocarbons. Examples of such other hydrocarbons which can be present in the feed at a level of 0.001 to 99.999 weight % include, but are not limited to, ethylene, propylene, 1-butene, 2-butene, isobutylene, 1-pentene, 2-pentene, methyl-1-butenes (such as 2-methyl-l-butene), methyl-2-butenes (such as 2-methyl-2-butene), 1-hexene, 2-hexene, 3-hexene, methyl-1-pentenes, 2,3-dimethyl-1-butene, 1-heptene, 2-heptene, 3-heptene, methyl-1-hexenes, methyl-2-hexenes, methyl-3-hexenes, dimethylpentenes, ethylpentenes, octenes, methylheptenes, dimethylhexenes, ethylhexenes, nonenes, methyloctenes, dimethylheptenes, ethylheptenes, trimethylhexenes, cyclopentene, cyclohexene, methylcyclopentene, cycloheptene, methylcyclohexene, dimethylcyclopentenes, ethylcyclopentenes, cyclooctenes, methylcycloheptenes, dimethylcyclohexenes, ethylcyclohexenes, trimethylcyclohexenes, methylcyclooctenes, dimethylcyclooctenes, ethylcylcooctenes, benzene, toluene, ethylbenzene, styrene, xylenes, methane, ethane, propane, butane, methylpropane, methylbutane, dimethylbutane, pentanes, hexanes, and the like, and combinations of two or more than two of these hydrocarbons.

Furthermore, the fluid feed can contain 0 to about 10 weight % hydrogen, and up to 1000 parts per million volume (ppmv) of carbon monoxide.

Also, small amounts of other components typically found in olefin streams can be present also.

The selective hydrogenation process is generally carried out by contacting a feed stream comprising at least one highly unsaturated hydrocarbon and molecular hydrogen with the catalyst of this invention which is generally contained in a fixed bed.

In a typical commercial acetylene removal unit, the highly unsaturated hydrocarbon is hydrogenated to an unsaturated hydrocarbon and a portion of the unsaturated hydrocarbon is hydrogenated to a saturated hydrocarbon. During the course of operation, the amount of low level impurities (such as CO, sulfur compounds, polyenenes, . . . ) in the feed which help regulate the reaction can vary. If their concentration decreases, the catalyst can start to hydrogenate the unsaturated hydrocarbon. If enough of the unsaturated hydrocarbon is hydrogenated the exotherm produced can propagate a runaway hydrogenation reaction.

If the impurity level increases in the ARU, the reaction temperature will have to be increased to maintain the hydrogenation of the highly unsaturated hydrocarbon. The runaway threat becomes apparent when the plant corrects itself and the impurity level decreases. At this point the reactor is running at too high of a temperature and a runaway hydrogenation reaction becomes probable.

A sulfur compound can be added to the feed stream continuously, it can be added to the feed stream for a period sufficient to affect the catalyst and then discontinued or it can be introduced in to the feedstream on the occurrence of finding that a particular product (such as ethane) in the ARU exit stream has reached a critical that, if not reduced, can cause a runaway reaction. To affect the catalyst is defined as the activity of the catalyst being decreased.

The temperature necessary for the selective hydrogenation process of this invention depends largely upon the activity of the catalyst composition, the hydrocarbon feed composition, and the desired extent of hydrogenation. Generally, reaction temperatures in the range of from about 10° C. to about 300° C., preferably about 20° C. to about 250° C., and most preferably 25° C. to 200° C. can be used. A suitable reaction pressure generally is in the range of about 15 to about 2,000 pounds per square inch gauge (psig), preferably 50 to about 1,500 psig, and most preferably 100 to 1,000 psig. The gas hourly space velocity (GHSV) of the hydrocarbon feed can vary over a wide range. Typically, the space velocity of the feed will be in the range of about 500 to about 40,000 liters of hydrocarbon feed per liter of catalyst hour, more preferably about 1,000 to about 30,000 liters/liter catalyst hour.

The following examples are presented to further illustrate this invention and should not be construed as unduly limiting the scope of this invention.

### EXAMPLE I

This example illustrates the preparation of the catalyst compositions used in the present invention.

Catalyst A was a commercially available material designated G83A obtained from United Catalysts Incorporated located in Louisville, Ky. Catalyst A contained 0.018 to 0.02 weight percent palladium on aluminum oxide support.

Catalyst B was a commercially available material designated G83C obtained from the same source. Catalyst B contained 0.016 to 0.018 weight percent palladium and 0.048 weight percent silver on aluminum oxide support.

Catalyst C was made as described in U.S. Pat. No. 5,585,318, incorporated here by reference. Catalyst C contained 0.02 weight percent palladium, 0.3 weight percent potassium and 0.3 weight percent fluoride on aluminum oxide support.

### Example II

About 20 cc (23 g) of each of the above described catalysts was placed in a stainless steel reactor tube having a 0.5 inches inner diameter and a length of about 18 inches. The catalyst (resided in the middle of the reactor; both ends of the reactor were packed with 6 ml of 3 mm glass beads) was reduced at 38° C. for 1 hour under hydrogen at 12 l/hour at 200 psi. Thereafter, a hydrocarbon feed containing approximately (all by weight unless otherwise noted) hydrogen, 2%; methane, 22%; propylene, 23%; ethylene, 51%; acetylene, 5000 ppm; propadiene 5000 ppm; methylacetylene 5000 ppm; and carbon monoxide, 300 ppm (by volume) was continuously introduced into the reactor. The feed rate was 900 ml per minute at 200 psi. The reaction was allowed to runaway. During the runaway the heat of hydrogenation builds up such that the reactor temperature exceeds 250° F. The catalyst was then allowed to cool to room temperature, about 75° F. before data collection was started. The reactor temperature was gradually increased to the desired reaction temperature, samples of the formed product were analyzed at various time intervals by means of a gas chromatograph.

Next, the reactor was heated to about 55-85° C. and a large concentration of carbonyl sulfide (COS) (approximately 300 mL at standard temperature and pressure (STP) of 5000 parts per million parts by volume (ppmv) COS in nitrogen) was passed over the catalyst. COS concentrations were then regulated by substituting part of the hydrogen flow (flowing at 12 liters/hour at 200 psig) with a blend of COS and hydrogen (COS/H₂ blend). The flow rates were set to deliver the hydrocarbon-containing fluid, hydrogen, and COS/H₂ blend to obtain a concentration of 12 parts COS per million parts by volume of total feed. Data was then collected as described above.

After the COS data were collected, the reactor was heated to 200° F. and purged with nitrogen at 1.4L/min at 200 psi for 1 hour. After the reactor cooled, the hydrogen and hydrocarbon were reintroduced and COS data was collected as described above.

In the results shown in Table I, T₁ is referred to as the cleanup temperature, that is the temperature at which the acetylene concentration falls below 20 ppm. T₂ is referred to as runaway temperature, that is the temperature at which 3 weight % of ethylene is hydrogenated to ethane (at this temperature the heat produced by the hydrogenation cannot be dissipated and ethylene hydrogenation proceeds at a rate at which it can not stop by itself). ΔT is the difference between T₂ and T₁. The ΔT is a measure of ethylene selectivity which can also be viewed as a window of operability.

TABLE I

| | | COS | T₁ | T₂ | | Extra_{Δ} T |
|---|---|---|---|---|---|---|
| Run | Catalyst | 12 ppmv | ° C. | ° C. | _{Δ}T | T_{2(COS)}− T_{2(no COS)} |
| 101 | A | none | 115 | 147 | 32 | — |
| 102 | A | 12 ppmv | 129 | 170 | 41 | 23 |
| 103 | A | after | 117 | 156 | 39 | — |
| 201 | B | none | 130 | 170 | 40 | — |
| 202 | B | 12 ppmv | 184 | 210 | 26 | 40 |
| 203 | B | after | 142 | 188 | 46 | — |
| 301 | C | none | 134 | 167 | 33 | — |
| 302 | C | 12 ppmv | 150 | 204 | 54 | 37 |
| 303 | C | after | 134 | 204 | 68 | — |

The extra ΔT demonstrates the benefit of the added sulfur compound. The addition of the COS is shown to have raised the T₂ by 23° C. (Runs 101-102), 40° C. (Runs 201-202) and 37° C. (Runs 301-302) thereby stopping a runaway reaction. Even after the COS is removed, the ΔT is still enhanced as the T₁ returns to normal.

### EXAMPLE 3

The reactions carried out in this example were similar to those in Example 2. About 20 cc (23 g) of each of catalyst was placed in a stainless steel reactor tube having a 0.5 inches inner diameter and a length of about 18 inches. The catalyst (resided in the middle of the reactor; both ends of the reactor were packed with 6 ml of 3 mm glass beads) was reduced at 38° C. for ½ hour under hydrogen at 12 l/hour at 200 psi. Thereafter, a hydrocarbon feed containing approximately (all by weight unless otherwise noted) hydrogen, 2% methane, 25%; ethylene, 57%; acetylene, 3300 ppm; and carbon monoxide, 250 ppm (by volume) was continuously introduced into the reactor. The feed rate was 1100 ml per minute at 200 psi. The reactor temperature was increased to the desired reaction temperature, samples of the formed product were analyzed at various time intervals by means of a gas chromatograph. Once the reaction was lined out with 5-10 ppm acetylene surviving, a 300 mL slug of 1000 ppmv COS in nitrogen was introduced. The heater temperature was held constant while the exit system was monitored for acetylene and ethane. This data is shown in Table 4.

TABLE IV

| | | | time after | Ethane | Acetylene |
|---|---|---|---|---|---|
| | | temperature ^{a} | COS injection | make | in exit |
| Run | Cat | ° F. | minutes | ppm | stream ppm |
| 401 | A | 101.7 | −20 | 5925 | 9 |
| | | 98 | 10 | 412 | 3425 |
| | | 95 | 40 | 321 | 1786 |
| | | 95 | 75 | 329 | 1129 |
| 402 | B | 115 | −20 | 3289 | 9 |
| | | 114 | 10 | 294 | 3470 |
| | | 111.8 | 40 | 279 | 2548 |
| | | 111.8 | 60 | 289 | 1696 |

From runs 401 and 402 it is readily seen that prior to the injection of COS, the catalyst has hydrogenated virtually all of the acetylene and is over hydrogenating the ethylene. Even 60 minutes after the COS slug, the reactor has not fully recovered to its pre-COS operations. Thus demonstrating that a COS injection can stop the hyrogenation reaction.

That which is claimed is:

1. A process comprising contacting a fluid which comprises highly unsaturated hydrocarbon, in the presence of hydrogen and a sulfur compound, with a catalyst composition under a condition effective to convert said highly unsaturated hydrocarbon to less unsaturated hydrocarbon wherein said catalyst composition comprises palladium, an inorganic support and, optionally, a component chosen from the group consisting of silver and alkali metal fluoride.

2. A process according to claim 1 wherein said sulfur compound is contained in said fluid.

3. A process according to claim 1 wherein said sulfur compound is injected into said fluid.

4. A process according to claim 1 wherein said sulfur compound is present in said fluid for a portion of time sufficient to affect the catalyst.

5. A process according to claim 1 wherein said sulfur compound is injected into said fluid for a portion of time sufficient to affect the catalyst.

6. A process according to claim 1 wherein said sulfur compound is COS.

7. A process according to claim 1 wherein contacting the catalyst with a fluid containing carbonyl sulfide takes place prior to contacting the fluid which comprises a highly unsaturated hydrocarbon, in the presence of hydrogen, with the catalyst composition.

8. A process according to claim 1 wherein contacting the catalyst with a fluid containing carbonyl sulfide takes place during contacting the fluid which comprises a highly unsaturated hydrocarbon, in the presence of hydrogen, with the catalyst composition.

9. A process according to claim 3 wherein the highly unsaturated hydrocarbon comprises acetylene, the less saturated hydrocarbon comprises ethylene and contacting the catalyst with a fluid containing carbonyl sulfide.

10. A process according to claim 1 wherein said fluid further comprises an alkene.

11. A process according to claim 10 wherein said alkene is selected from the group consisting of ethylene, propylene, butenes, and combinations of two or more thereof.

12. A process according to claim 11 wherein said alkene is ethylene.

13. A process according to claim 10 wherein said inorganic support material is selected from the group consisting of alumina, silica, titania, zirconia, zinc aluminate, zinc titanate, and combinations of two or more thereof.

14. A process according to claim 10 wherein said palladium is present as skin on said support.

15. A process according to claim 10 wherein said highly unsaturated hydrocarbon is selected from the group consisting of diolefins, alkynes, and combinations of two or more thereof.

16. A process according to claim 15 wherein said highly unsaturated hydrocarbon is selected from the group consisting of propadiene, methyl acetylene 1,2-butadiene, 1,3-butadiene, isoprene, 1,2-pentadiene, 1,3-pentadiene, 1,4-pentadiene, 1,2-hexadiene, 1,3-hexadiene, 1,4-hexadiene, 1,5-hexadiene, 2-methyl-1,2-pentadiene, 2,3-dimethyl-1,3-butadiene, heptadienes, methylhexadienes, octadienes, methylheptadienes, dimethylhexadienes, ethylhexadienes, trimethylpentadienes, methyloctadienes, dimethylheptadienes, ethyloctadienes, trimethylhexadienes, nonadienes, decadienes, undecadienes, dodecadienes, cyclopentadienes, cyclohexadienes, methylcyclopentadienes, cycloheptadienes, methylcyclohexadienes dimethylcyclopentadienes, ethylcyclopentadienes, dicyclopentadiene, acetylene, propyne, 1-butyne, 2-butyne, 1-pentyne, 2-pentyne, 3-methyl-1-butyne, 1-hexyne, 1-heptyne, 1-octyne, 1-nonyne, 1-decyne, and combinations of two or more thereof.

17. A process according to claim 16 wherein said highly unsaturated hydrocarbon is selected from the group consisting of propadiene, 1,3-butadiene, 1,3-pentadiene, 1,4-pentadiene, isoprene, 1,3-cyclopentadiene, dicyclopentadiene, acetylene, propyne, 1-butyne, 2-butyne, 1-pentyne, 2-pentyne, 3-methyl-1-butyne, 1-hexyne, 1-heptyne, 1-octyne, 1-nonyne, 1-decyne, and combinations of two or more thereof.

18. A process according to claim 15 wherein said highly unsaturated hydrocarbon is acetylene.

19. A process according to claim 16 wherein said highly unsaturated hydrocarbon comprises acetylene.

20. A process according to claim 17 wherein said highly unsaturated hydrocarbon comprises acetylene.

21. A process comprising contacting a fluid stream which comprises an alkene and a highly unsaturated hydrocarbon, in the presence of hydrogen, with a catalyst composition under a condition effective to convert said highly unsaturated hydrocarbon to a less unsaturated hydrocarbon wherein a finite amount of a sulfur compound in a range up to about 0.005 weight % is added to the fluid stream for a time sufficient to affect the catalyst and wherein
said catalyst composition comprises palladium, an inorganic support and, optionally, a component chosen from the group consisting of silver and alkali metal fluoride;
said alkene is selected from the group consisting of ethylene, propylene, butenes, and combinations of two or more thereof;
said inorganic support is selected from the group consisting of alumina, silica, titania, zirconia, zinc aluminate, zinc titanate, magnesium aluminate, and combinations of two or more thereof;
said catalyst composition contains about 0.001 to about 3 weight % palladium; optionally, about 0.002 to about 10 weight % silver; optionally about 0.05 to about 1.5 weight % alkali metal and fluorine (chemically bound as alkali metal fluoride);
said highly unsaturated hydrocarbon is selected from the group consisting of diolefins, alkynes, and combinations of two or more thereof.

22. A process according to claim 21 wherein said highly unsaturated hydrocarbon is selected from the group consisting of propadiene, propadiene, methyl acetylene, 1,2-butadiene, 1,3-butadiene, isoprene, 1,2-pentadiene, 1,3-pentadiene, 1,4-pentadiene, 1,2-hexadiene, 1,3-hexadiene, 1,4-hexadiene, 1,5-hexadiene, 2-methyl-1,2-pentadiene, 2,3-dimethyl-1,3-butadiene, heptadienes, methylhexadienes, octadienes, methylheptadienes, dimethylhexadienes, ethylhexadienes, trimethylpentadienes, methyloctadienes, dimethylheptadienes, ethyloctadienes, trimethylhexadienes, nonadienes, decadienes, undecadienes, dodecadienes, cyclopentadienes, cyclohexadienes, methylcyclopentadienes, cycloheptadienes, methylcyclohexadienes dimethylcyclopentadienes, ethylcyclopentadienes, dicyclopentadiene, acetylene, propyne, 1-butyne, 2-butyne, 1-pentyne, 2-pentyne, 3-methyl-1-butyne, 1-hexyne, 1-heptyne, 1-octyne, 1-nonyne, 1-decyne, and combinations of two or more thereof.

23. A process according to claim 21 wherein said support is alumina; catalyst composition contains about 0.001 to about 2 weight % palladium which is distributed as skin on said alumina; and said highly unsaturated hydrocarbon is selected from the group consisting of acetylene, propadiene, methyl acetylene, 1,3-butadiene, 1,3-pentadiene, 1,4-pentadiene, isoprene, 1,3-cyclopentadiene, dicyclopentadiene, and combinations of two or more thereof.

24. A process according to claim 23 wherein said alkene is ethylene.

25. A process according to claim 24 wherein said highly unsaturated hydrocarbon comprises acetylene.

26. A selective hydrogenation process comprising contacting a fluid stream which comprises ethylene and a highly unsaturated hydrocarbon, in the presence of hydrogen, with a catalyst composition under a condition effective to convert said highly unsaturated hydrocarbon to a less unsaturated hydrocarbon wherein
said catalyst composition comprises palladium, alumina and, optionally, a component chosen from the group consisting of silver and alkali metal fluoride;
said catalyst is contacted with a finite amount of a sulfur compound added to the fluid stream for a time sufficient to affect the catalyst;
said highly unsaturated hydrocarbon comprises acetylene;
said process is carried out at a temperature in the range of about 30 to about 200° C. and under a pressure in the range of about 15 to about 2000 psig.
